(19) United States
(12) Patent Application Publication
(10) Pub. No.: US 2007/0010802 A1
(43) Pub. Date: Jan. 11, 2007

(54) Surgical instrument

(75) Inventor: Jean-Albert Despres, Souesmes (FR)

Correspondence Address: LEYDIG VOIT & MAYER, LTD, 700 THIRTEENTH ST. NW, SUITE 300, WASHINGTON, DC, 20005-3960, US

(73) Assignee: CYRIL DE UTHEMANN, Vandoeuvres (CH)

(21) Appl. No.: 11/478,327

(22) Filed: Jun. 30, 2006

(30) Foreign Application Priority Data
Jul. 4, 2005 (FR) 05 07 079

(51) Int. Cl. A61B 17/00

(52) U.S. Cl. 606/1

(57) ABSTRACT

The present invention relates to an instrument such as a surgical instrument including an insert bearing an identification code of the surgical instrument, arranged in a closed cavity in the surgical instrument.

The invention relates to an instrument, such as a surgical instrument of a predetermined type, defined by its shape and its function out of a number of instruments of different types.

The instruments of this type that are known can be recognized or identified by the persons who are supposed to manipulate them by their exterior visual appearance, but they do not lend themselves to sorting that can be done automatically.

The invention aims to propose instruments of which the type can be recognized automatically.

In order to attain this aim, an instrument according to the invention is characterized by the fact that it is provided with an insert bearing an identification code of the instrument, arranged in a closed cavity made beforehand in the instrument.

According to another characteristic of the invention, the insert is produced out of a material that is relatively opaque to X rays, and the instrument is made out of a material that is relatively transparent to these X rays so that it can be recognized using X rays.

According to yet another characteristic of the invention, the code elements of the insert are constituted by the specific shape of the insert.

According to yet another characteristic of the invention, the insert is produced out of a material such as brass or an alloy based on brass or any other metal that is more opaque to X rays than the metal constituting the instrument.

The invention will be better understood and other aims, characteristics, details and advantages of it will appear more clearly in the course of the following explanatory description in reference to the appended diagrammatic drawings given only by way of example illustrating an embodiment of the invention and in which:

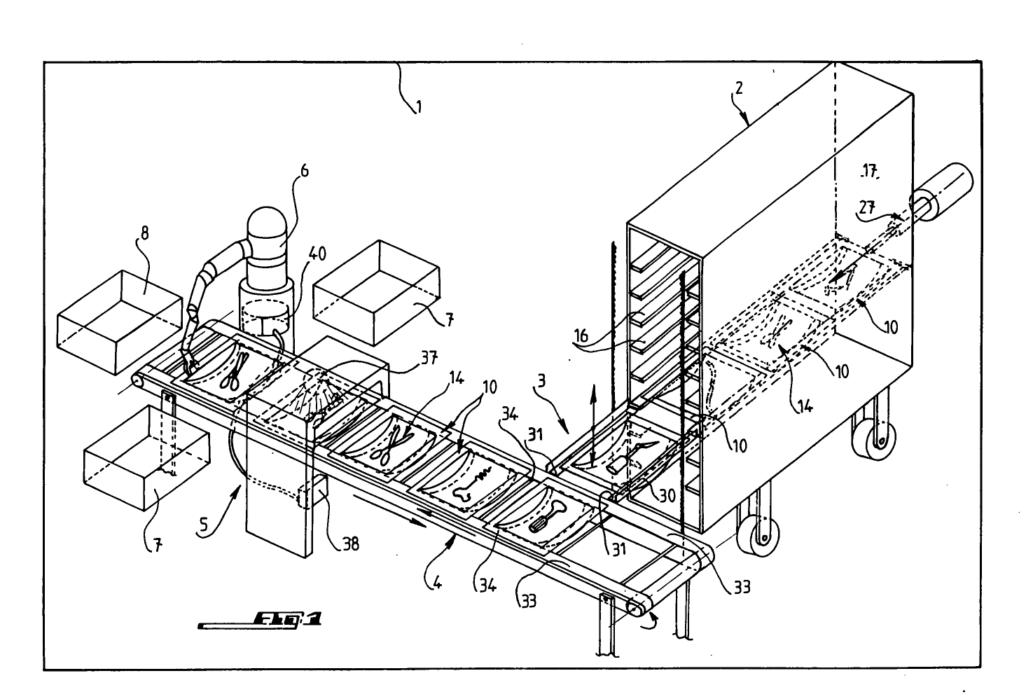

FIG. 1 is a diagrammatic view in perspective of an installation according to the invention;

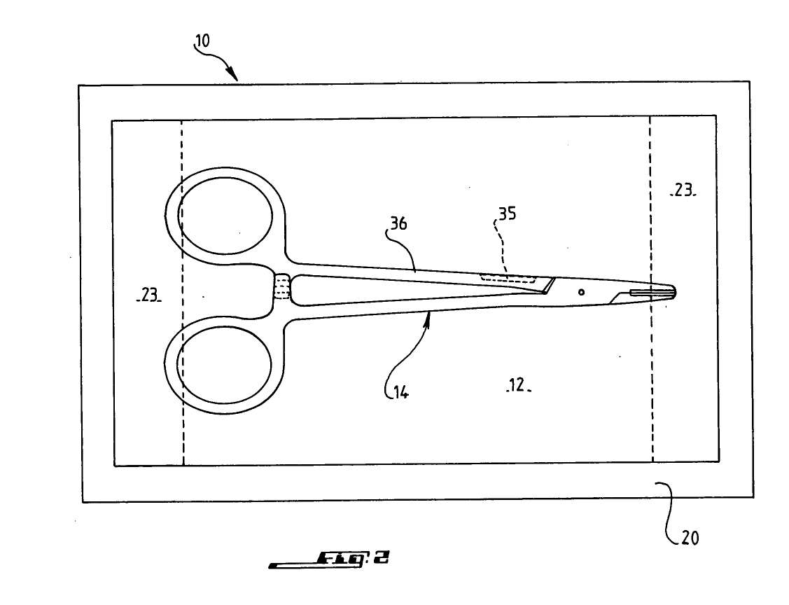

FIG. 2 is a top view of an instrument support tray according to the invention;

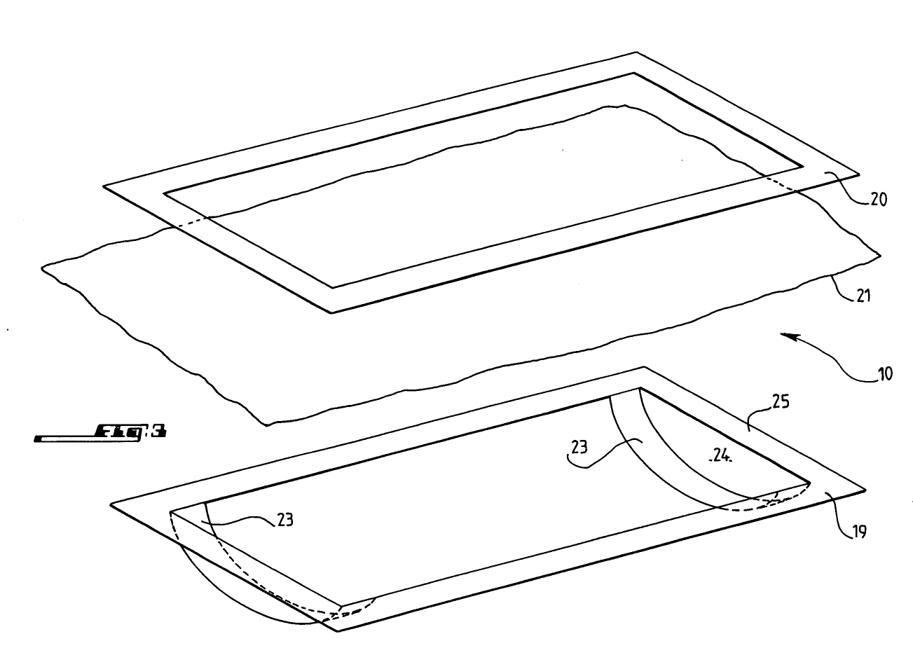

FIG. 3 is an exploded view of a support tray according to FIG. 2; and

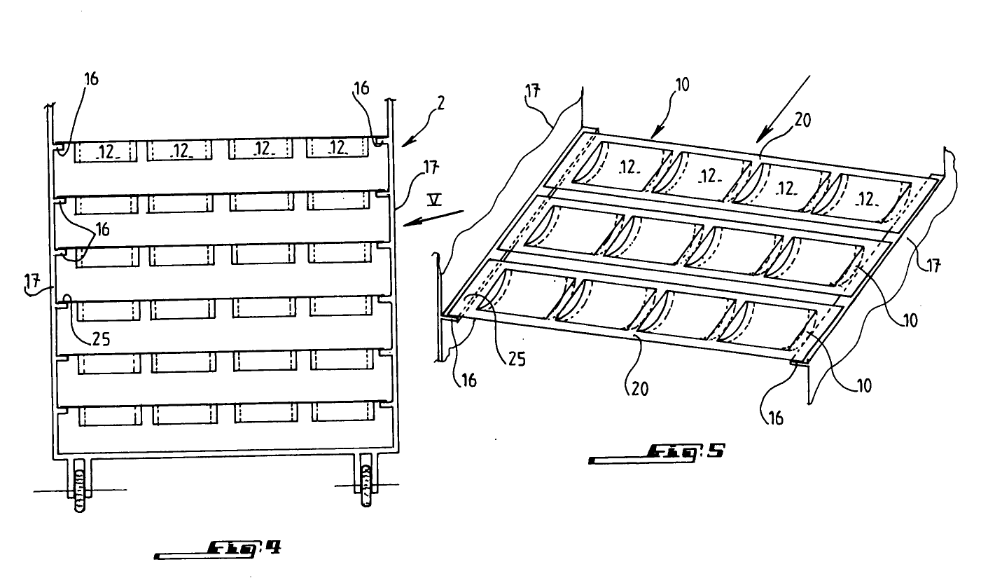

FIGS. 4 and 5 illustrate an advantageous configuration of trays according to the invention and of a storage staging.

Given that the advantage of the invention lies in the fact that the instruments to which the invention relates allow automatic recognition of their type by means of X rays, they will be described hereafter in the context of an automatic surgical instrument sorting process and installation. Of course, the description is given only by way of example; however, in a general manner, the invention can be used for any operation involving a specific selection and stowing of instruments for implementation of the operation.

In reference to FIG. 1, it is observed that an installation according to the invention for sorting of the instruments necessary for a surgical operation essentially entails, arranged inside of enclosure 1 with a pure atmosphere, that is to say of the most perfect cleanliness possible, essentially a device for storage of all of the surgical instruments likely to be used for the different possible surgical operations, in the clean state, device 3 for transfer of the instruments to conveyor 4 intended for transporting them to station 5 for recognition of the nature or type of instruments, and a mechanism for stowing the instruments after their recognition in containers 7 each of which is intended for holding the instruments that are to be used for a pre-determined operation, container 8 being provided for receiving instruments considered not to be in accordance with the requirements established for surgical operations.

Device 2 for storage of all of the instruments is executed in the example in the form of a carriage with a certain number of levels 9 for holding each of a number of trays 10, 3 trays in the example represented, each of which can have a number of chambers 12 for housing of surgical instrument 14. In this specific case, for reasons of simplification of the drawings, each tray has only one chamber. Trays 10 of each level 9 of storage carriage 2 are supported by support elements in the form of slide rails 16 each attached to lateral wall 17 of the carriage oriented in the direction of conveyor 4.

In reference to FIG. 3, it is observed that each tray 10 is composed of two superposed frames made of a material that is easy to clean such as stainless steel, namely lower frame element 19 with a general rectangular shape and upper frame 20 of complementary shape which can be attached on the lower frame by means in the form of clips (not represented) that are capable of squeezing the upper frame onto the lower frame so that replaceable element 21, advantageously a single-use element, made of a flexible material transparent to X rays such as paper or fabric, can be inserted at two of its opposite edges between the corresponding edges of the two frames in order to form chamber 12 for housing of instrument 14 and thus the bottom of the tray. For easy formation and maintaining of the chambers, lower frame 19 is provided with support elements 23 in the form of arches at the site of each longitudinal end. It is advantageous for the chamber to be closed at each longitudinal end by vertical wall 24 forming the space between the frame and the corresponding arch 23.

As seen in FIG. 1, each tray 10 can be moved in carriage 2, perpendicularly to its longitudinal axis, resting by short sides 25 on slide rails 16 of the carriage.

The arrangement for transfer of trays 10 of each stage of the carriage entails, in order to push the trays out of the carriage, pushing device 27 mounted so as to be vertically mobile at the rear of carriage 2, so that it can be positioned at each level 9 of the carriage. The device has a pushing piston of a hydraulic jack that, during its out movement, pushes the last tray and thus moves all of the trays in the direction of the conveyor.

Transfer device 3 moreover has, in front of storage carriage 2 for trays 10, frame 30 for transfer of trays 10 from carriage 2 to conveyor 4, which can be moved vertically so that it can receive the trays of each storage level 9 of the carriage in order to transport them then to the height of conveyor 4 so that the trays can be put on the conveyor. More precisely, in the example represented, the transfer frame essentially has two slide rails 31, each capable of being aligned, in a position for receiving a tray from level or stage 9, with slide rail 16 of the level, so that trays 10 can be moved under the effect of pushing device 27 from rails 16 of the carriage to rails 31 of transfer frame 30. The transfer frame is dimensioned in such a way as to successively transfer trays 10 to conveyor 4.

Conveyor 4 is shown in FIG. 1 as produced in the form of an endless conveyor belt with essentially two parallel flat belts 33 for the transport of trays 10, which are connected by crosspieces 34.

In order to ensure the transfer of trays 10 from transfer frame 30 to conveyor belts 33, support rails 31 of the transfer frame extend to the point that they are over the conveyor, and its tray support portion, in its position before transfer, can be lowered in order to allow placement of the carriages over the belts of the conveyor situated below. Then they move away laterally and return to their position for receiving another tray.

Conveyor 4 transports the trays received from storage carriage 2 by the intermediary of transfer frame 3 to station 5 for recognition of the instruments placed in chambers 12 of trays 10.

Concerning surgical instruments, which have different functions and shapes, each of them is provided with an identification code that can be recognized by station 5 so that the station can distinguish the instruments brought in by conveyor 4 according to their specific type. More precisely, the identification code of each instrument is marked in any appropriate form on insert 35 that is placed in an appropriate cavity machined in the instrument and then closed. FIG. 2 shows as an example surgical pliers with insert 35 incorporated in one of its branches noted 36.

The station for recognition of the instruments is advantageously an X ray reader in the form of a portal through which conveyor 4 passes and which has, arranged above conveyor 4, X ray source 37, while a receiver of the rays passing through instrument 14 is arranged below the conveyor.

So that it is possible to recognize the type of instrument according to its insert, the elements of the insert that form the identification code must be less transparent to X rays than the material constituting the instrument. This code could reside in the shape of the insert or of marks provided on it or else the shape or placement of a notch made in the insert. Since inserts of this type are known, it is not necessary to describe them in more detail. The inserts are advantageously produced out of a material that is relatively opaque to X rays. They could be made out of brass or an alloy based on brass, while the instruments are made out of stainless steel. In a general manner, for the choice of materials, the heavier the atoms constituting the material are, that is to say the higher their atomic weight, the more they absorb X rays and therefore the more opaque to X rays the material is. In order to ensure reliable recognition of the instruments, it is necessary for the instruments to be presented at the recognition station always in a clearly defined position.

After the recognition of the instruments by reading of their inserts using X rays, stowing apparatus 6 grasps the instruments and stows them in containers 7 in the form of boxes, under the orders of information processing device 40.

This device has operation protocols in its memory, one protocol for each type of operation, indicating the instruments that are to be used in the course of the operation, if applicable in their order of use. Given that each type of operation corresponds to a box 7, the instruments to be stowed in this box are indicated by the protocol established for this operation. For this purpose, information processing device 40 first identifies, according to the signal that it has just received from reader 38 of the recognition station, the type of instrument that has just been examined and determines by referring to the different protocols which operation and thus which box 7 an instrument of this type is intended for. Then, it gives the order to stowing device 6 to grasp the identified instrument in the tray and to stow it in the appropriate box.

By comparing the instruments stowed in a box with those appearing in the protocol, the information processing device knows at any time the “filling” state of each box 7. If it establishes that a box is finished, that is to say contains all the instruments necessary for the operation in consideration, the box is closed, for example, by putting its cover on.

The invention also provides for the possibility of separating instruments considered unfit for use from the circuit of utilization, by placing them in discard box 8. There could be various reasons for this measure, for example, a worn instrument, an instrument that is impossible to identify or because an instrument is soiled.

Concerning stowing station 6, apparatuses capable of functioning in the manner described above are generally known so that it is not necessary to describe precisely the apparatus used in the context of the invention. It is appropriate to indicate that such an apparatus has a robot arm capable of grasping the instruments in their tray and placing them then in the appropriate boxes as a function of the instructions received from the information processing device.

Concerning the functioning of the invention and the running of the process as well as the different steps of the process, they proceed from the description just given. It goes without further specifying that for each transfer of a tray onto the conveyor, the conveyor is stopped for a brief moment of time necessary for placing the tray. The stops of the conveyor for loading of the trays and the process of recognition of the instruments by the recognition station as well as the stowing of the instruments are coordinated by the information processing device.

The description of the invention just given has been given only as an example, and multiple modifications can be made without leaving the scope of the invention. In order to increase the storage capacity of carriage 2, each tray 10 could have four chambers as shown by FIGS. 4 and 5. The arrow symbolizes the action of the mechanism for moving a tray towards conveyor 3.

1. A surgical instrument of a pre-determined type, distinguished by shape and function from other surgical instruments and including an insert bearing an identification code of the surgical instrument, in a closed cavity in the surgical instrument.

2. The surgical instrument according to claim 1, wherein the insert is a material that is relatively opaque to X rays, and the surgical instrument is a material that is relatively transparent to X rays so that the insert can be recognized using X rays.

3. The surgical instrument according to claim 2, wherein the identification code of the insert includes elements selected from shape of the insert, shape of a notch in the insert, and marks of the insert.

4. The surgical instrument according to claim 2, wherein the insert is brass or an alloy based on brass, and the surgical instrument is stainless steel.

5. The surgical instrument according to claim 3, where the insert is brass or an alloy based on brass, and the surgical instrument is stainless steel.
